(19) United States
(12) Patent Application Publication
(10) Pub. No.: US 2023/0398381 A1
(43) Pub. Date: Dec. 14, 2023

(54) MULTIPARAMETRIC OPTIMIZATION FOR ULTRASOUND PROCEDURES

(71) Applicant: INSIGHTEC LTD., Tirat Carmel (IL)

(72) Inventors: Shuki Vitek, Haifa (IL); Kobi Vortman, Haifa (IL)

(21) Appl. No.: 18/252,133

(22) PCT Filed: Nov. 17, 2021
(86) PCT No.: PCT/IB2021/000795

§ 371 Date: May 8, 2023

Related U.S. Application Data
(60) Provisional application No. 63/115,267, filed on Nov. 18, 2020.

(51) Int. Cl. A61N 7/02; A61B 90/00; A61B 34/10

(52) U.S. Cl. CPC A61N 7/02; A61B 90/37; A61B 34/10; A61B 2034/104; A61N 2007/0095

(57) ABSTRACT

Focused-ultrasound systems and methods involve simultaneously determining multiple ultrasound parameters (e.g., applied acoustic power, frequency, phases, position, activation pattern, beam shape, wave shape, etc.) associated with the transducer array for generating optimal treatment effects both at the target region (e.g., causing a sufficient temperature increase for tissue necrosis to occur) and non-target region (e.g., having a clinically insignificant temperature increase to avoid damage to the non-target tissue). A computational model may simulate the treatment effects (e.g., the temperature, peak intensity, focus shape, location of the hot spot, etc.) of these ultrasound parameters on the target and/or non-target regions. Based on the simulation results, the computational model may simultaneously determine the optimal values of the multiple ultrasound parameters—i.e., the values that, while not necessarily optimal for each individual parameter considered in isolation, nonetheless produce optimal overall treatment effects at the target and non-target

### RELATED APPLICATION

This application claims the benefit of and priority to U.S. Provisional Patent Application No. 63/115,267, filed Nov. 18, 2020, the entire disclosure of which is hereby incorporated by reference.

### TECHNICAL FIELD

The present invention relates, generally, to focused-ultrasound procedures, and more particularly to systems and methods for multiparametric optimization of such procedures.

### BACKGROUND

Focused ultrasound (i.e., acoustic waves having a frequency greater than about 100 kiloHertz) can be used to image or therapeutically treat internal body tissues within a patient. For example, ultrasound waves may be used in applications involving ablation of tumors, targeted drug delivery, disruption of the blood-brain barrier (BBB), lysing of clots, and other surgical procedures. The noninvasive nature of ultrasound procedures is particularly appealing for ablating or disrupting target tissue (e.g., diseased tissue or BBB) surrounded by or neighboring healthy tissue or organs because the effects of ultrasound energy can be confined to a well-defined target region. Ultrasonic energy may be focused as a beam to a zone having a cross-section of only a few millimeters due to relatively the short wavelengths (e.g., as small as 1.5 millimeters (mm) in cross-section at one MegaHertz (1 MHz)) utilized. Moreover, because acoustic energy generally penetrates well through soft tissues, intervening anatomy often does not impose an obstacle to defining a desired focal zone. Thus, ultrasonic energy may be focused at a small target in order to ablate diseased tissue while minimizing damage to surrounding healthy tissue.

To focus ultrasonic energy at a desired target, drive signals may be sent to an acoustic transducer having multiple distributed transducer elements such that constructive interference occurs at the focal zone. At the target, sufficient acoustic intensity may be delivered to heat tissue until necrosis occurs, i.e., until the tissue is destroyed. Preferably, non-target tissue along the acoustic path through which the acoustic energy passes (the “pass zone”) outside the focal zone is exposed to low intensity acoustic beams and thus will be heated only insignificantly, if at all, thereby minimizing damage to tissue outside the focal zone.

Typically, ultrasonic energy is delivered according to a treatment plan, often based on a predefined model of the target and the patient's anatomy. During treatment, the treatment effects (e.g., the temperature) at the target are monitored using, for example, a magnetic resonance imaging (MRI) apparatus. If the measured temperature is below the desired target temperature for necrosis, the power of the ultrasonic waves transmitted from the transducer is increased. Another parameter that affects the temperature is the ultrasound transmission frequency. Thus, another approach to increasing the temperature at the target is via adjustment of the transmission frequency of the transducer array (see, e.g., U.S. Patent Publication No. 2019/0009109). When the system is operated in a long-burst mode (e.g., having a high number of cycles or continuous waves), correction of the phase shifts between different transducer elements may allow signals from all transducer elements to converge constructively at the target.

Each of these approaches can therefore determine an optimal value of a single transducer parameter (e.g., amplitude, phase or frequency) for reducing any deviation between the measured temperature and the planned (i.e., desired) target temperature, thereby optimizing one or more of the treatment effects (e.g., the temperature at the target region). But other treatment effects that occur during the ultrasound procedure require consideration. For example, undesired hot spots in the pass zone should be minimized in order to avoid damage to the healthy tissue. The transducer parameter values chosen to optimize one treatment effect (e.g., the temperature at the target) may not be optimal for another effect (e.g., hot spots in the pass zone). In addition, because the tissue properties in the pass zone and the target region may dynamically change during treatment (e.g., due to application of the acoustic energy), the ultrasound parameters determined based on tissue properties prior to sonication may not provide optimal clinical results as the treatment proceeds.

Accordingly, there is a need for an approach that simultaneously evaluates multiple treatment effects resulting from multiple ultrasound parameters associated with the transducer array and dynamically adjust values of these parameters so as to optimize overall treatment efficacy during the ultrasound procedure.

### SUMMARY

Various embodiments of the present invention provide focused-ultrasound approaches that involve simultaneously determining multiple ultrasound parameters (e.g., applied acoustic power, frequency, phases, position, activation pattern, beam shape, wave shape, etc.) associated with the transducer array for generating optimal treatment effects both at the target region (e.g., causing a sufficient temperature increase for tissue necrosis to occur) and non-target region (e.g., having a clinically insignificant temperature increase to avoid damage to the non-target tissue), as well as systems for implementing such approaches. In one embodiment, a computational model is implemented to simulate the treatment effects (e.g., the temperature, peak intensity, focus shape, location of the hot spot, etc.) of these ultrasound parameters on the target and/or non-target regions. The computational model may simulate the interactions of the ultrasound beam with the patient's target tissue and/or intervening tissue located in the pass zone with various beam powers, frequencies, shapes, activation patterns, etc. using, for example, conventional finite-element analysis. In addition, the simulation may be based on a detailed tissue model as acquired by an imaging apparatus (e.g., computer tomography, ultrashort echo-time (TE), MRI, etc.); the model generally includes multiple tissue types or layers (e.g., for ultrasound focusing into the skull, layers of cortical bone, bone marrow, and soft brain tissue) and characterizes their respective anatomic and/or material properties. Based on the simulation results, the computational model may simultaneously determine the optimal values of the multiple ultrasound parameters—i.e., the values that, while not necessarily optimal for each individual parameter considered in isolation, nonetheless produce optimal overall treatment effects at the target and non-target regions (e.g., maximal energy absorption and highest intensity (or energy density) at the target and minimal energy deposition at the non-target region).

During treatment, the transducer is operated in accordance with the computationally determined parameter values. In addition, at least some of the treatment effects at the target and non-target regions are simultaneously monitored in real time. In one embodiment, a parameter-optimizing approach is implemented to compare the measured treatment effects against the treatment effects predicted by the computational model and then simultaneously adjust at least some of the ultrasound parameters so as to improve the measured treatment effects. For example, the parameter-optimizing approach may define a cost function representing deviations of the measured treatment effects from the predicted effects and update the values of two or more ultrasound parameters until the cost function is minimized. The transducer can then be operated based on the updated parameter values. In various embodiments, the treatment effects are continuously monitored and provided as feedback for adjusting the values of the ultrasound parameters throughout the entire ultrasound procedure; this can maintain optimal overall clinical effects as the treatment proceeds.

As used herein, the terms “optimal,” “optimizing,” “maximum,” “maximizing”, etc. generally involve a substantial improvement (e.g., by more than 10%, more than 20%, or more than 30%) over treatment undertaken with optimization, e.g., in accordance with the prior art, but do not necessarily imply achievement of the best theoretically possible ultrasound parameter values or treatment effects. Rather, optimizing the ultrasound parameter values involves reconciling competing treatment priorities, and determining and selecting the best overall parameter values practically identifiable within the limitations of the utilized technology and method. The invention recognizes that multiple treatment effects can be greatly affected by multiple ultrasound parameters, and that clinical results can be significantly improved by simultaneously and continuously updating the ultrasound parameter values during the procedure.

In addition, because interactions between the acoustic waves and tissue is non-linear, compensation for the interactions may be performed in a non-linear manner by, for example, measuring the errors and changing the ultrasound parameter values. In addition, the term “insignificant clinical temperature increase” means having no undesired temperature increase that is considered significant by clinicians, e.g., the onset of damage to tissue or other clinically adverse effect, whether temporary or permanent.

Accordingly, in a first aspect, the invention relates to a system for delivering ultrasound energy to a target region. In various embodiments, the system comprises an ultrasound transducer comprising a plurality of transducer elements for generating a focal zone of acoustic energy at the target region; at least one measurement system for measuring a plurality of treatment effects at the target region and/or a non-target region; and a controller configured to (a) computationally determine values of ultrasound parameters associated with at least some of the transducer elements and predict the treatment effects at the target region and/or the non-target region; (b) operate the ultrasound transducer based at least in part on the determined values of ultrasound parameters; (c) cause the measurement system(s) to measure at least some of the treatment effects at the target region and/or the non-target region; (d) compare the measured treatment effects against the predicted treatment effects; (e) based on the comparison, computationally update the values of at least some of the ultrasound parameters; and (f) operate the ultrasound transducer based at least in part on the updated values of ultrasound parameters.

In some embodiments, the measurement system(s) comprise at least one of a magnetic resonance imaging device, an ultrasonography device, a positron emission tomography device, a single-photon emission computed tomography device, or a computer tomography device. The controller may be further configured to compute deviations of the measured treatment effects from the predicted treatment effects and define a cost function based on the deviations. For example, the controller may be further configured to simultaneously and iteratively update the values of at least some of ultrasound parameters until a value of the cost function is minimized or below a predetermined threshold.

In various embodiments, the controller is further configured to repeat steps (c)-(f). The ultrasound parameters may comprise one or more (or all) of power, frequency, phase, position and/or an activation pattern. The treatment effects may comprise one or more (or all) of a temperature at the target region and/or the non-target region, a peak acoustic intensity at the target region, a focus shape, a location of a hot spot or a tissue perfusion rate at the target region and/or the non-target region.

The system may further include an imaging system for acquiring images of the target region and/or the non-target region, in which case the controller may be further configured to analyze the acquired images for determining at least one of an anatomic characteristic or a material characteristic of tissue in the target region and/or the non-target region. The anatomic characteristic may comprise at least one of a type, a property, a structure, a thickness or a density associated with the issue. The material characteristic may comprise at least one of an energy absorption of the tissue at a specific frequency or a speed of sound. In various embodiments, the ultrasound transducer is a phased-array transducer.

In another aspect, the invention pertains to a method of delivering ultrasound energy from an ultrasound transducer comprising a plurality of transducer elements to a target region. In various embodiments, the method comprises (a) computationally determining values of ultrasound parameters associated with at least some of the transducer elements and predicting treatment effects at the target region and/or a non-target region; (b) operating the ultrasound transducer based at least in part on the determined values of ultrasound parameters; (c) electronically measuring at least some of the treatment effects at the target region and/or the non-target region; (d) comparing the measured treatment effects against the predicted treatment effects; (e) based on the comparison, computationally updating the values of at least some of the ultrasound parameters; and (0 operating the ultrasound transducer based at least in part on the updated values of ultrasound parameters.

In some embodiments, steps (c)-(f) are repeated. The treatment effects may be measured by at least one of a magnetic resonance imaging device, an ultrasonography device, a positron emission tomography device, a single-photon emission computed tomography device, or a computer tomography device. The method may include the step of computing deviations of the measured treatment effects from the predicted treatment effects and computationally defining a cost function based on the deviations. The values of at least some of the ultrasound parameters may be simultaneously and iteratively updated until a value of the cost function is minimized or below a predetermined threshold. The ultrasound parameters may comprise a power, a frequency, a phase, a position and an activation pattern or a subset of these parameters.

The predicted treatment effects may comprise one or more of the temperature at the target region and/or the non-target region, the peak acoustic intensity at the target region, the focus shape, the location of a hot spot or a tissue perfusion rate at the target region and/or the non-target region.

As used herein, the term “substantially” means±10%, and in some embodiments, ±5% of the peak intensity. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A illustrates a focused ultrasound system in accordance with various embodiments.

FIG. 1B illustrates an exemplary imaging system in accordance with various embodiments.

FIG. 2 is a flow chart depicting a representative treatment and optimization procedure in accordance with embodiments hereof.

### DETAILED DESCRIPTION

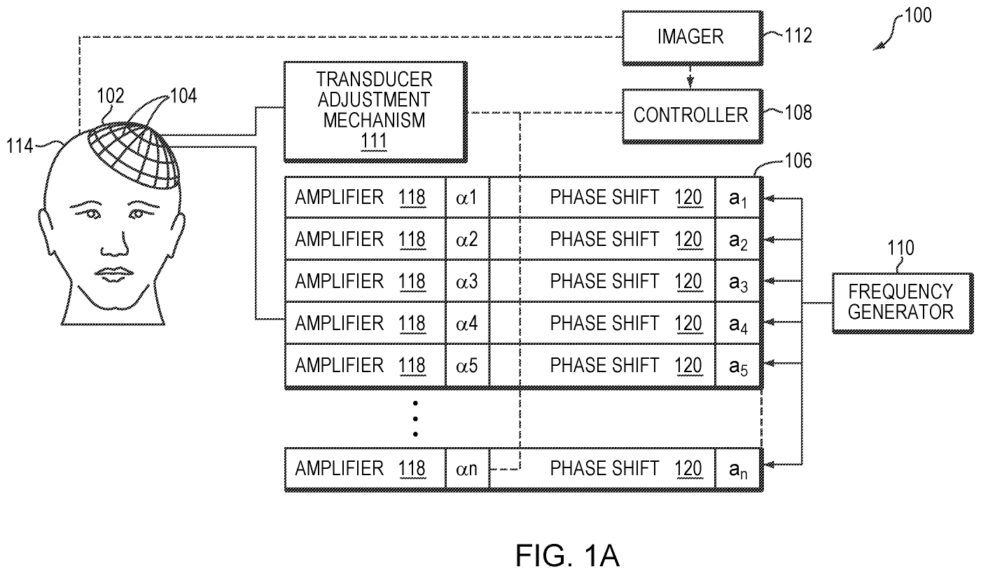

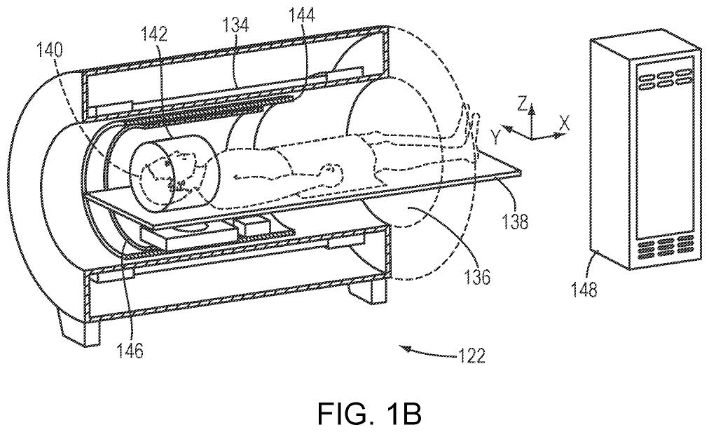

Refer first to FIG. 1A, which illustrates an exemplary ultrasound system 100 for focusing ultrasound onto a target region 101 in a patient. The system 100 can shape the ultrasonic energy in various ways, producing, for example, a point focus, a line focus, a ring-shaped focus, or multiple foci simultaneously. In various embodiments, the system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) shape suitable for placing it on the surface of a skull or a body part other than the skull, or may include one or more planar or otherwise shaped sections. Its dimensions may vary, depending on the application, between millimeters and tens of centimeters. The transducer elements 104 of the array 102 may be piezoelectric ceramic, capacitive micromachined ultrasonic transducer (CMUT) or microelectromechanical systems (MEMS) elements, and may be mounted in silicone rubber or any other material suitable for damping the mechanical coupling between the elements 104. Piezo-composite materials, or generally any materials shaped in a manner facilitating conversion of electrical energy to acoustic energy, may also be used. To assure maximum power transfer to the transducer elements 104, the elements 104 may be configured for electrical resonance, matching input impedance. In addition, the system may include a transducer-adjustment mechanism 111 (e.g., a motor, a gimbal, or other manipulator that permits mechanical and/or electrical adjustment of the orientation (e.g., an angle or a position) and/or translation (if desired) of ultrasound beams emitted from the transducer array 102 and/or individual transducer elements 104 therein. For example, the transducer-adjustment mechanism 117 may physically rotate the transducer elements 104 around one or more axes thereof and/or move the elements 104 with respect to the target 101 to a desired location. Alternatively or additionally, the transducer-adjustment mechanism 111 may adjust the orientation of the ultrasound beam electronically by changing the beam path via the beamformer 106, which responsively alters the relative phases of the transducer elements so as to change the beam path or acoustic beam shape, for example, by deactivating some of the transducer elements. In some embodiments, the transducer-adjustment mechanism 111 is responsive to a communication from the controller 108.

The transducer array 102 is coupled to the beamformer 106, which drives the individual transducer elements 104 so that they collectively produce a focused ultrasonic beam or field. For n transducer elements, the beamformer 106 may contain n driver circuits, each circuit including or consisting of an amplifier 118 and a phase shift circuit 120; drive circuit drives one of the transducer elements 104. The beamformer 106 receives a radio frequency (RF) input signal, typically in the range from 0.1 MHz to 4.0 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual transducer elements 104 of the transducer array 102 at the same frequency (or in some embodiments, different group of elements at different frequencies), but at different phases and/or different amplitudes.

The amplification or attenuation factors α₁-αₙ and the phase shifts a₁-aₙ imposed by the beamformer 106 serve to transmit and focus ultrasonic energy through inhomogeneous tissue (e.g., the patient's skull or different tissues located in the acoustic paths of ultrasound beams from the transducer elements to the target region or “path zones”) onto the target region (e.g., a region in the patient's brain). Via adjustments of the amplification factors and/or the phase shifts, a desired shape and intensity of a focal zone may be created at the target region.

The necessary amplification factors and phase shifts may be computed by the controller 108, which may provide the relevant computational functions through software, hardware, firmware, hardwiring, or any combination thereof. For example, the controller 108 may utilize a general-purpose or special-purpose digital data processor programmed with software in a conventional manner, and without undue experimentation, to determine the frequency, phase shifts and/or amplification factors of the transducer elements 104. In certain embodiments, the controller computation is based on information about the characteristics (e.g., structure, thickness, density, etc.) of intervening tissues located between the transducer 102 and the target 101 (e.g., the pass zone) and their effects on propagation of acoustic energy. In various embodiments, such information is obtained from an imager 112 (such as a magnetic resonance imaging (MRI) device, a computer tomography (CT) device, a positron emission tomography (PET) device, a single-photon emission computed tomography (SPECT) device, or an ultrasonography device) or a combination of multiple imagers. Image acquisition may be three-dimensional (3D) or, alternatively, the imager 112 may provide a set of two-dimensional (2D) images suitable for reconstructing a three-dimensional image of the target region 101 and/or other regions (e.g., the region surrounding the target 101, the region in the pass zone located between the transducer and the target 101, or another target region). Image-manipulation functionality may be implemented in the imager 112, in the controller 108, or in a separate device.

FIG. 1B illustrates an exemplary imager—namely, an MRI apparatus 112. The apparatus 112 may include a cylindrical electromagnet 134, which generates the requisite static magnetic field within a bore 136 of the electromagnet 134. During medical procedures, a patient is placed inside the bore 136 possibly on a movable support table 138. An anatomic region of interest 140 (e.g., the patient's head) may be positioned within an imaging region 142 wherein the electromagnet 134 generates a substantially homogeneous field. A set of cylindrical magnetic field gradient coils 144 may also be provided within the bore 136 and surrounding the patient. The gradient coils 144 generate magnetic field gradients of predetermined magnitudes, at predetermined times, and in three mutually orthogonal directions. With the field gradients, different spatial locations can be associated with different precession frequencies, thereby giving an MR image its spatial resolution. An RF transmitter coil 146 surrounding the imaging region 142 emits RF pulses into the imaging region 142 to cause the patient's tissues to emit magnetic-resonance (MR) response signals. Raw MR response signals are sensed by the RF coil 146 and passed to an MR controller 148 that then computes an MR image, which may be displayed to the user. Alternatively, separate MR transmitter and receiver coils may be used. Images acquired using the MRI apparatus 112 may provide radiologists and physicians with a visual contrast between different tissues and detailed internal views of a patient's anatomy that cannot be visualized with conventional x-ray technology.

The MRI controller 148 may control the pulse sequence, i.e., the relative timing and strengths of the magnetic field gradients and the RF excitation pulses and response detection periods. The MRI controller 148 may be combined with the transducer controller 108 into an integrated system control facility.

The MR response signals are amplified, conditioned, and digitized into raw data using a conventional image-processing system, and further transformed into arrays of image data by methods known to those of ordinary skill in the art. The image-processing system may be part of the MRI controller 148, or may be a separate device (e.g., a general-purpose computer containing image-processing software) in communication with the MRI controller 148 and/or the transducer controller 108. Because the response signal is tissue- and temperature-dependent, it can be processed to identify the treatment target region (e.g., a tumor to be destroyed by heat) 101 in the image, as well as to compute a temperature map from the image. Further, the acoustic field resulting from ultrasound application may be monitored in real time, using, e.g., thermal MRI or MR-based acoustic radiation force imaging. Thus, using MRI data, the ultrasound transducer 102 may be driven so as to focus ultrasound into (or near) the target region 101, while the temperature of the target and surrounding tissues and/or the acoustic field intensity are being monitored.

As described above, MR imaging can provide a non-invasive means of quantitatively monitoring in vivo temperatures. This is particularly useful in MR-guided thermal therapy (e.g., MR-guided focused ultrasound (MRgFUS) treatment), where the temperature of the target region 101 should be continuously monitored in order to assess the progress of treatment and to correct for local differences in ultrasound absorption, reflection, scattering and conduction thereby avoiding damage to tissues surrounding the target. Monitoring (e.g., measurement and/or mapping) of the temperature is generally based on MR imaging (referred to as MR thermometry or MR thermal imaging) in conjunction with suitable image-processing software.

Among various methods available for MR thermometry, the PRF shift method is often the method of choice due to its linearity with respect to temperature change, near-independence from tissue type, and the high spatial and temporal resolution of temperature maps obtained therewith. The PRF shift method is based on the phenomenon that the MR resonance frequency of protons in water molecules changes linearly with temperature (with a constant of proportionality that, advantageously, is relatively constant among tissue types). Since the frequency change with temperature is small, only −0.01 ppm/° C. for bulk water and approximately −0.0096 to −0.013 ppm/° C. in tissue, the PRF shift is typically detected with a phase-sensitive imaging method in which the imaging is performed twice: first to acquire a baseline PRF phase image prior to a temperature change and then to acquire a second phase image after the temperature change—i.e., a treatment image—thereby capturing a small phase change that is proportional to the change in temperature. A map of temperature changes may then be computed from the (reconstructed, i.e., real-space) images by determining, on a pixel-by-pixel basis, phase differences between the baseline image and the treatment image, and converting the phase differences into temperature differences based on the PRF temperature dependence while taking into account imaging parameters such as the strength of the static magnetic field and echo time (TE) (e.g., of a gradient-recalled echo).

In various embodiments, prior to an ultrasound treatment procedure, the MRI apparatus 112 acquires one or more images of the target 101 and/or non-target regions (e.g., regions located in the pass zone between the transducer 104 and the target 101). The acquired MR images provide accurate locational information of the target/non-target regions for the purpose of treatment planning, as well as baseline phase maps for determining the temperature at the target and/or non-target regions. In general, the MRI thermometry sequence starts with acquisition of baseline images (e.g., at the start of the sonication); a new phase image may be acquired every 2 to 5 seconds.

In some embodiments, based on the locational information of the target/non-target regions, the values of the ultrasound parameters (e.g., the position, relative phase shift, frequency, etc.) associated with one or more transducer elements 104 that result in a focused beam at the identified target region 101 may be computed. This step generally involves a computational model that utilizes a reverse-ray tracking approach by taking into account the geometry as well as the position and orientation of the ultrasound transducer 102 relative to the target region 101, as well as any a-priori knowledge and/or image-derived information about the intervening tissues. In some embodiments, different imaging apparatus are involved in determining the relative position of the target with respect to the transducer elements. For example, the orientations and locations of the transducer elements may be obtained using, e.g., a time-of-flight approach in the ultrasound system, whereas the spatial characteristics of the target region may be acquired using MRI. As a consequence, it may be necessary to register coordinate systems in different imaging modalities prior to computing the expected amplitude and/or phase associated with each transducer element. Exemplary registration approaches are provided, for example, in U.S. Pat. No. 9,934,570, the entire disclosure of which is hereby incorporated by reference.

In addition, the computational model may compute the ultrasound parameter values (e.g., the positions, amplification factors and/or the relative phase shifts) associated with the transducer elements for creating a focus having desired focusing properties (e.g., a maximal peak intensity, a minimal focal area, a desired focus shape, etc.) at the target region. For example, the positions of the transducer elements may be adjusted to achieve the lowest f-number of the ultrasound phased array (and thereby the smallest focal area); and the relative phases and amplitudes of the ultrasound waves transmitted from the transducer elements may be varied to achieve the desired focus shape and peak intensity.

To determine the optimal ultrasound parameter values, in some embodiments, the computational model includes anatomic characteristics (e.g., the type, property, structure, thickness, density, etc.) and/or material characteristics (e.g., the energy absorption of the tissue at a specific frequency or the speed of sound) of the target tissue. For example, because liver tissue is highly dynamic and vascular, it may be desired to apply ultrasound waves having a relatively high power with a short sonication time when treating a liver tumor. In addition, the computational model may include the anatomic/material characteristics of the intervening tissue located in the pass zone between the transducer and the target region in order to predict and correct for beam aberrations resulting therefrom. For example, the intervening tissue may be defined as more than one region, each region corresponding to a particular transducer element or grouping of the transducer elements. In addition, each region of the intervening tissue may have a different sensitivity level to the deposited acoustic energy and/or a different absorption rate of the deposited acoustic energy based on the anatomic/material characteristics associated with the tissue therein. Thus, to avoid overheating intervening tissue having a higher sensitivity level and/or a higher energy absorption rate, the transducer element(s) corresponding thereto may be deactivated (at least periodically). Accordingly, the computational model may determine activation and deactivation patterns of at least some of the transducer elements 104 based on the anatomic/material characteristics of the intervening tissue along the path zone, thereby avoiding damage to the healthy tissue.

The anatomic characteristics of the target and/or intervening tissue may be acquired using the imager 112. For example, based on the acquired images of the anatomic region of interest, a tissue model characterizing the material characteristics of the target and/or non-target regions may be established. The tissue model may take the form of a 3D table of cells corresponding to the voxels representing the target and/or non-target tissue; the values of the cells represent characteristics of the tissue, such as the speed of sound, that are relevant to aberrations that occur when the beam traverses the tissue, or the tissue sensitivity level to the deposited acoustic energy. The voxels are obtained tomographically by the imager and the type of tissue that each voxel represents can be determined automatically by conventional tissue-analysis software. Using the determined tissue types and a lookup table of tissue parameters (e.g., speed of sound and/or tissue sensitivity level by type of tissue), the 3D table of the tissue model may be populated. Further detail regarding creation of a tissue model that identifies the speed of sound, heat sensitivity and/or thermal energy tolerance of various tissues may be found in U.S. Patent Publication No. 2012/0029396, the entire disclosure of which is hereby incorporated by reference.

Deactivation of some of the transducer elements, however, may cause distortion of the focused beam, thereby degrading the intensity, uniformity and shape of the focus. Thus, in some embodiments, the computational model can account for the effects resulting from deactivation of the transducer elements when updating the ultrasound parameter values. Further, the peak intensity/power in the focal zone may also be affected by the frequency of the ultrasound waves transmitted from the transducer array. Adjusting the ultrasound frequency, however, may result in changes in other parameters (e.g., the steering angle of the focused beam), which may also affect the peak acoustic intensity. Accordingly, the computational model may determine an optimal transmission frequency by simulating the effects of all (or at least some) frequency-dependent parameters on the peak acoustic intensity/power in the focal zone. Approaches to determining the optimal frequency of the ultrasound transducer are provided, for example, in U.S. Patent Publication No. 2020/0205782, the entire disclosure of which is hereby incorporated by reference.

In some embodiments, based on the computed values of the ultrasound parameters and the anatomic and/or material characteristics of the target/non-target tissue, the computational model can computationally predict ultrasound energy delivered to the target region and/or non-target regions, the conversion of ultrasound energy or pressure into heat and/or tissue displacement at the target region and/or non-target regions, and/or the propagation of the induced effects through the tissue. Consequently, the treatment effects (e.g., temperatures at the target and non-target regions, the peak intensity and/or the focus shape at the target region, the location of hot spots in the non-target region, etc.) can be predicted using the computational model. In various embodiments, the computational model adjusts the values of the ultrasound parameters until the treatment effects are optimized, e.g., (a) all clinical effects are acceptable or (b) the parameters produce the best clinical result obtainable for each treatment effect in light of the other treatment effects. An example of all clinical effects being acceptable is the predicted temperature at the target region exceeding a desired target temperature for necrosis and the absence of any clinically deleterious hot spots in the non-target region. In cases where mutually optimizing all parameters produces one or more unacceptable clinical effects, the optimization criterion can be relaxed so that all clinical effects are acceptable and the deviation from the best mutual parameter optimization is tolerated.

Typically, the simulation takes the form of (or includes) differential equations that describe the temperature evolution and/or heating process in tissue, taking into account, for example, heat transfer through thermal conduction or blood perfusion, metabolic heat generation, and/or absorption of energy applied to the tissue—e.g., the well-known Pennes bioheat transfer equation. The differential equations, supplemented by suitable initial and/or boundary conditions (e.g., a known temperature profile at the beginning of treatment, or a fixed temperature at a boundary of the zone of interest), may be straightforwardly solved numerically (or, in certain cases, analytically) to simulate temperature evolution and heating processes in the zone of interest, and thereby predict the temperature as a function of time and/or space (or at one or more selected discrete points in time and space). Approaches to simulating the sonications and their effects on the tissue are provided, for example, in U.S. Patent Publication No. 2015/0359603, the entire disclosure of which is hereby incorporated by reference.

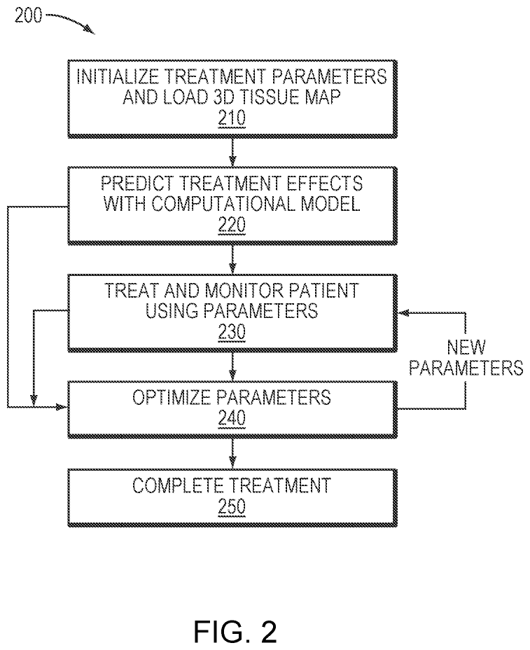

A representative method 200 in accordance with embodiments of the invention is shown in FIG. 2. The treatment procedure begins (step 210) with initializing and operating the transducer in accordance with the parameter values determined using the computational model. A tissue model, e.g., a 3D voxel representation of the target tissue and surrounding regions annotated for tissue sensitivities, is loaded into the controller 108 (FIG. 1A). In step 220, the controller 108 predicts the treatment effect of operating the transducer with the initial treatment parameters generated in step 210. As the patient is treated, multiple treatment effects (e.g., the temperature, peak intensity, focus shape, location of the hot spot, tissue perfusion, etc.) are substantially simultaneously monitored in real time (step 230) by a measurement system implemented within the controller 108. For example, MRI thermometry may measure the temperature increase associated with each voxel of the 3D tissue map representing the target and/or non-target tissue; MR-ARFI may measure the tissue displacement resulting from acoustic pressure at the target; and ultrasound reflection detection may measure the intensity of the ultrasound that is reflected from the target/non-target regions. These data are gathered by the measurement system of the controller 108. In step 240, the controller 108 executes a parameter optimization to compare the measured treatment effects against the treatment effects predicted by the computational model and adjust multiple ultrasound parameter values so as to improve the treatment effects. For example, parameter optimization may be based on a cost function for deviations of the measured treatment effects from the predicted effects. In one implementation, the cost function is simply defined as:

$$Cost = {\sum }_{i = 1}^{n} {w}_{i} ( {T}_{m , i} - {T}_{p , i} )$$

where T_{m,i} and T_{p,i} represent the measured and predicted temperatures, respectively, at voxel i of the MRI image, and wᵢ represents a weight assigned to voxel i. In one implementation, the weight function is assigned based on the tissue sensitivity level and/or its absorption rate of acoustic energy. For example, voxels representing non-target tissue having a higher sensitivity level or acoustic absorption rate may be assigned larger weight factors compared to voxels representing non-target tissue having a lower sensitivity level or acoustic absorption rate.

In various embodiments, based on the cost function, parameter optimization may simultaneously and iteratively update the values of two or more ultrasound parameters until the value of the cost function is minimized or below a predetermined threshold. Subsequently, step 230 is repeated and the transducer array 102 is operated based on the updated parameter values.

In step 230, the treatment effects are continuously monitored and fed back to the parameter optimization so that the values of the ultrasound parameters may be adjusted throughout the ultrasound procedure. Accordingly, various embodiments monitor multiple treatment effects at the target/non-target regions during treatment and dynamically adjust multiple ultrasound parameters that are related to deposition of the acoustic energy at the target and/or non-target tissue so as to optimize multiple treatment effects during the entire ultrasound procedure until completion (step 250).

It should be noted that the cost function in Eq. (1) is exemplary only; any suitable functions may be used and are thus within the scope of the invention. For example, the cost function may be a nonlinear function such that a larger penalty is given when, for example, the temperatures of the voxels at a non-target region having a high sensitivity level exceed the predicted temperatures. In addition, the cost function may include other terms such as the deviation of the measured peak intensity from the predicted peak intensity, the deviation of the focus shape from the predicted focus shape, etc. Alternatively, mutual optimization of multiple parameters may be achieved using linear programming (such as the simplex method) or nonlinear programming (using extensions to the simplex method such as the generalized reduced gradient method).

In general, functionality for determining and updating the optimal values for the transducer elements as described above, whether integrated within a controller of the imager, and/or an ultrasound system, or provided by a separate external controller, may be structured in one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C #, BASIC, various scripting languages, and/or HTML. Additionally, the software can be implemented in an assembly language directed to the microprocessor resident on a target computer (e.g., the controller); for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

In addition, the term “controller” used herein broadly includes all necessary hardware components and/or software modules utilized to perform any functionality as described above; the controller may include multiple hardware components and/or software modules and the functionality can be spread among different components and/or modules.

Further, the terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. For example, instead of MR-based thermometry or ARFI, any non-invasive imaging technique capable of measuring the (physical or therapeutic) treatment effects of the acoustic beam at the focus may generally be used. Accordingly, the described embodiments are to be considered in all respects as only illustrative and not restrictive.

What is claimed is:

1. A system for delivering ultrasound energy to a target region, the system comprising:
an ultrasound transducer comprising a plurality of transducer elements for generating a focal zone of acoustic energy at the target region;
at least one measurement system for measuring a plurality of treatment effects at the target region and/or a non-target region; and
a controller configured to:
(a) computationally determine values of ultrasound parameters associated with at least some of the transducer elements and predict the treatment effects at the target region and/or the non-target region;
(b) operate the ultrasound transducer based at least in part on the determined values of ultrasound parameters;
(c) cause the at least one measurement system to measure at least some of the treatment effects at the target region and/or the non-target region;
(d) compare the measured treatment effects against the predicted treatment effects;
(e) based on the comparison, computationally update the values of at least some of the ultrasound parameters; and
(f) operate the ultrasound transducer based at least in part on the updated values of ultrasound parameters.

2. The system of claim 1, wherein the at least one measurement system comprises at least one of a magnetic resonance imaging device, an ultrasonography device, a positron emission tomography device, a single-photon emission computed tomography device, or a computer tomography device.

3. The system of claim 2, wherein the controller is further configured to compute deviations of the measured treatment effects from the predicted treatment effects and define a cost function based on the deviations.

4. The system of claim 3, the controller is further configured to simultaneously and iteratively update the values of at least some of ultrasound parameters until a value of the cost function is minimized or below a predetermined threshold.

5. The system of claim 1, wherein the controller is further configured to repeat steps (c)-(f).

6. The system of claim 1, wherein the ultrasound parameters comprise a power, a frequency, a phase, a position and an activation pattern.

7. The system of claim 1, wherein the treatment effects comprise at least one of a temperature at the target region and/or the non-target region, a peak acoustic intensity at the target region, a focus shape, a location of a hot spot or a tissue perfusion rate at the target region and/or the non-target region.

8. The system of claim 1, further comprising an imaging system for acquiring images of the target region and/or the non-target region.

9. The system of claim 8, wherein the controller is further configured to analyze the acquired images for determining at least one of an anatomic characteristic or a material characteristic of tissue in the target region and/or the non-target region.

10. The system of claim 9, wherein the anatomic characteristic comprises at least one of a type, a property, a structure, a thickness or a density associated with the issue.

11. The system of claim 9, wherein the material characteristic comprises at least one of an energy absorption of the tissue at a specific frequency or a speed of sound.

12. The system of claim 1, wherein the ultrasound transducer is a phased-array transducer.

13. A method of delivering ultrasound energy from an ultrasound transducer comprising a plurality of transducer elements to a target region, the method comprising:
(a) computationally determining values of ultrasound parameters associated with at least some of the transducer elements and predicting treatment effects at the target region and/or a non-target region;
(b) operating the ultrasound transducer based at least in part on the determined values of ultrasound parameters;
(c) electronically measuring at least some of the treatment effects at the target region and/or the non-target region;
(d) comparing the measured treatment effects against the predicted treatment effects;
(e) based on the comparison, computationally updating the values of at least some of the ultrasound parameters; and
(f) operating the ultrasound transducer based at least in part on the updated values of ultrasound parameters.

14. The method of claim 13, further comprising repeating steps (c)-(f).

15. The method of claim 13, wherein the treatment effects are measured by at least one of a magnetic resonance imaging device, an ultrasonography device, a positron emission tomography device, a single-photon emission computed tomography device, or a computer tomography device.

16. The method of claim 13, further comprising the step of computing deviations of the measured treatment effects from the predicted treatment effects and computationally defining a cost function based on the deviations.

17. The method of claim 16, further comprising the step of simultaneously and iteratively updating values of at least some of the ultrasound parameters until a value of the cost function is minimized or below a predetermined threshold.

18. The method of claim 17, wherein the ultrasound parameters comprise a power, a frequency, a phase, a position and an activation pattern.

19. The method of claim 13, wherein the predicted treatment effects comprise at least one of a temperature at the target region and/or the non-target region, a peak acoustic intensity at the target region, a focus shape, a location of a hot spot or a tissue perfusion rate at the target region and/or the non-target region.
